(19) United States
(12) Patent Application Publication
(10) Pub. No.: US 2024/0399141 A1
(43) Pub. Date: Dec. 5, 2024

(54) MICRONEEDLES TO OVERCOME CONTACT RESISTANCE

(71) Applicant: Novocure GmbH, Root D4 (CH)

(72) Inventor: Richard Deslauriers, Woodbury, CT (US)

(21) Appl. No.: 18/622,108

(22) Filed: Mar. 29, 2024

Related U.S. Application Data
(60) Provisional application No. 63/493,571, filed on Mar. 31, 2023.

(51) Int. Cl. A61N 1/32; A61N 1/05

(52) U.S. Cl. CPC A61N 1/32; A61N 1/0502

(57) ABSTRACT

A transducer array, tumor treating field system, and method are herein disclosed. The transducer array comprises an electrode and a transfer member. The electrode has a first surface on a first skin-facing side and a second surface on an opposing outwardly-facing second side. The transfer member has a first surface on a first skin-facing side, and a second surface on an opposing outwardly-facing second side. The transfer member is located on the first skin-facing side of the electrode and has at least one protrusion extending in a skin-facing direction from the first surface and is configured to pierce a patient at a target area. The at least one protrusion has a base portion and an end portion.

### REFERENCE TO RELATED APPLICATIONS

The present patent application claims priority to U.S. Ser. No. 63/493,571 filed on Mar. 31, 2023, the entire contents of which are hereby incorporated herein by reference.

### BACKGROUND

Tumor Treating Fields (TTFields) are low intensity (e.g., 1-3 V/cm) alternating electric fields within the intermediate frequency range (e.g., 50 kHz to 1 MHz, such as 50-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the transducer arrays that make up each of these pairs are positioned on opposite sides of the body part that is being treated. More specifically, for the OPTUNE® system, one pair of electrodes of the transducer array is located to the left and right (LR) of the tumor, and the other pair of electrodes of the transducer array is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head. More recently, TTFields therapy has been approved as a concomitant therapy with chemotherapy for malignant pleural mesothelioma (MPM), and may find use in treating tumors in other parts of the body.

The transducer arrays are placed on the patient at target locations determined to have a high therapeutic value to treat the patient. The device is intended to be continuously worn by the patient for 2-4 days before removal for hygienic care and re-shaving (if necessary), followed by reapplication with a new set of arrays. However, hygienic care does not remove the keratinized layer of skin, which is a poor conductor and has a high resistance. Because patients use the device and go about their daily activities, the device may be used for an extended period of time during which the transducer array may have a tendency to move from the target location on the patient.

### SUMMARY OF THE DISCLOSURE

Thus, a need exists for a new and improved transducer array that does not move from the target location on the patient and penetrates through the keratinized layer of skin to increase conductivity. It is to such systems and methods of producing and using the same, that the present disclosure is directed.

The problem of the higher resistance provided by the keratinized layer of skin is solved by a transducer array, a tumor treating field system, and method of production and use thereof as described herein. In one embodiment, the present disclosure describes a transducer array. The transducer array comprises an electrode and a transfer member. The electrode has a first skin-facing side and an opposing outwardly-facing second side. The electrode comprises a first electrode surface on the first skin-facing side, and a second electrode surface on the opposing outwardly-facing second side. The transfer member comprising a support substrate having a first substrate surface on a first skin-facing side, wherein the support substrate is located on the first skin-facing side of the electrode and wherein the transfer member further comprises at least one protrusion extending in a skin-facing direction from the first substrate surface and is configured to pierce a patient at a target area, the at least one protrusion having a base portion and an end portion. In some embodiments, the at least one protrusion is a microneedle.

The details of one or more implementations of the subject matter described in this specification are set forth in the accompanying drawings and the description below. Other aspects, features and advantages of the subject matter will become apparent from the description, the drawings, and the claims.

### BRIEF DESCRIPTION OF THE DRAWINGS

The accompanying drawings, which are incorporated in and constitute a part of this specification, illustrate one or more implementation described herein. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function and a detailed description of like reference numerals varying only in alphabetical suffix may be omitted for conciseness unless otherwise specified. In the drawings:

FIG. 1 is an exemplary embodiment of a schematic diagram of electrodes as applied to living tissue.

FIG. 2 is an exemplary embodiment of an electronic device configured to generate an alternating electric field constructed in accordance with the present disclosure.

FIG. 3 is a block diagram of an exemplary embodiment of a transducer array constructed in accordance with the present disclosure.

FIG. 4A is a cross-sectional diagram of a first embodiment of the electrode element of the transducer array depicted in FIG. 3, taken along the lines 4-4′ in FIG. 3.

FIG. 4B is a cross-sectional diagram of a second embodiment of the electrode element of the transducer array depicted in FIG. 3, taken along the lines 4-4′ in FIG. 3.

FIG. 4C is a cross-sectional diagram of a third embodiment of the electrode element of the transducer array depicted in FIG. 3, taken along the lines 4-4′ in FIG. 3.

FIG. 4D is a cross-sectional diagram of a fourth embodiment of the electrode element of the transducer array depicted in FIG. 3, taken along the lines 4-4′ in FIG. 3.

FIG. 4E is a cross-sectional diagram of a fifth embodiment of the electrode element of the transducer array depicted in FIG. 3, taken along the lines 4-4′ in FIG. 3.

FIG. 4F is a cross-sectional diagram of a sixth embodiment of the electrode element of the transducer array depicted in FIG. 3, taken along the lines 4-4′ in FIG. 3.

FIG. 4G is a cross-sectional diagram of a seventh embodiment of the electrode element of the transducer array depicted in FIG. 3, taken along the lines 4-4′ in FIG. 3.

FIG. 5 is a perspective view of an exemplary embodiment of an electrode element constructed in accordance with the present disclosure.

FIG. 6 is an exemplary embodiment of a process of using the electronic apparatus and the transducer array of FIG. 2 to apply an alternating electric field to a patient in accordance with the present disclosure.

FIG. 7 is an exemplary embodiment of another process of using the electronic apparatus and the transducer array of FIG. 2 to apply an alternating electric field to a patient in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary-not exhaustive. Also, it is to be understood that the phraseology and terminology employed herein is for the purpose of description and should not be regarded as limiting.

Headings are provided for convenience only and are not to be construed to limit the disclosure in any manner. Embodiments illustrated under any heading or in any portion of the disclosure may be combined with embodiments illustrated under the same or any other heading or other portion of the disclosure. Any combination of the elements described herein in all possible variations thereof is encompassed by the disclosure unless otherwise indicated herein or otherwise clearly contradicted by context.

Unless otherwise required by context, singular terms shall include pluralities and plural terms shall include the singular.

All of the compositions, assemblies, systems, kits, and/or methods disclosed herein can be made and executed without undue experimentation in light of the present disclosure. Where a method claim does not specifically state in the claims or description that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

The use of the term “a” or “an” when used in conjunction with the term “comprising” in the claims and/or the specification may mean “one,” but is also consistent with the meaning of “one or more,” “at least one,” and “one or more than one.” The term “plurality” refers to “two or more.”

In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to collectively perform a task.

As used herein, all numerical values or ranges include fractions of the values and integers within such ranges and fractions of the integers within such ranges unless the context clearly indicates otherwise. The numerical ranges specified herein includes the endpoints, and all values, sub-ranges of values within the range, and fractions of the values and integers within said range. Thus, any two values within the range of 1 mm to 10 m, for example, can be used to set a lower and an upper boundary of a range in accordance with the embodiments of the present disclosure.

As used herein, the term TTField (or TTFields) refers to low intensity (e.g., 1-4 V/cm) alternating electric fields of medium frequencies (about 50 kHz-1 MHz, and more preferably from about 50 kHz-500 kHz) that when applied to a conductive medium, such as a human body, via electrodes may be used, for example, to treat tumors as described in U.S. Pat. No. 7,016,725, 7,089,054, 7,333,852, 7,565,205, 7,805,201, and 8,244,345 by Palti, the entire contents of which are hereby incorporated herein in their entirety, and in a publication by Kirson (see Eilon D. Kirson, et al., Disruption of Cancer Cell Replication by Alternating Electric Fields, Cancer Res. 2004 64:3288-3295). TTFields have been shown to have the capability to specifically affect cancer cells and serve, among other uses, for treating cancer. TTFields therapy is an approved mono-treatment for recurrent glioblastoma (GBM), and an approved combination therapy with chemotherapy for newly diagnosed GBM patients. Alternating electric fields can also be used to treat medical conditions other than tumors. For example, as described in U.S. Pat. No. 10,967,167 (which is incorporated herein by reference in its entirety), alternating electric fields can be used to increase the permeability of the blood brain barrier (BBB) so that, e.g., chemotherapy drugs can reach the brain.

As used herein, the term TTSignal is an electrical signal that, when received by electrodes applied to a conductive medium, such as a human body, causes the electrodes to generate the TTField described above. The TTSignal is often an AC, or alternating current waveform, electrical signal.

The term “transfer member” as used herein means an assembly comprising one or more protrusion connected to and extending from a surface of a support substrate.

The term “protrusion” as used herein means a slender rodlike instrument having one end forming a point and a length, when the surface of the transfer member is positioned on a user's skin, sufficient to pass through the epidermis of skin and terminate at the end forming the point within the skin's dermis and/or the skin's hypodermis without contacting the user's nerves. In some embodiments, the protrusion(s) are microneedle(s).

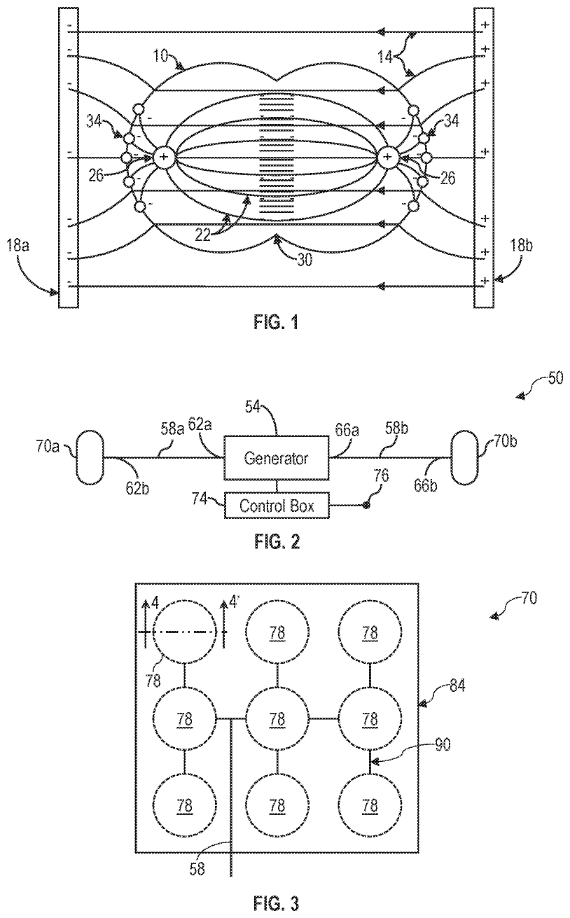

Referring now to the drawings and in particular to FIG. 1, shown therein is an exemplary embodiment of a dividing cell 10, under the influence of external TTFields, generally indicated as lines 14, generated by a first electrode 18a having a negative charge and a second electrode 18b having a positive charge. Further shown are microtubules 22 that are known to have a very strong dipole moment. This strong polarization makes the microtubules 22, as well as other polar macromolecules and especially those that have a specific orientation within the cell 10 or its surroundings, susceptible to electric fields. The positive charges of the microtubules 22 are located at two centrioles 26 while two sets of negative poles are at a center 30 of the dividing cell 10 and point of attachment 34 of the microtubules 22 to the cell membrane. The locations of the charges form sets of double dipoles and therefore are susceptible to electric fields of differing directions. In one embodiment, the cells go through electroporation, that is, DNA or chromosomes are introduced into the cells using a pulse of electricity to briefly open pores in the cell membranes.

Turning now to FIG. 2, the TTFields described above that have been found to advantageously destroy tumor cells may be generated by an electronic apparatus 50. FIG. 2 is a simple schematic diagram of the electronic apparatus 50 illustrating major components thereof. The electronic apparatus 50 includes an electric field generator 54 and a pair of conductive leads 58, including first conductive lead 58a and second conductive lead 58b. The first conductive lead 58a includes a first end 62a and a second end 62b. The second conductive lead 58b includes a first end 66a and a second end 66b. The first end 62a of the first conductive lead 58a is conductively attached to the electric field generator 54 and the first end 66a of the second conductive lead 58b is conductively attached to the electric field generator 54.

The electric field generator 54 is configured to supply power and generate desirable electric signals (TTSignals) in the shape of waveforms or trains of pulses as an output. The second end 62b of the first conductive lead 58a is connected to a first transducer array 70a and the second end 66b of the second conductive lead 58b is connected to a second transducer array 70b. Both of the first transducer array 70a and the second transducer array 70b are supplied with the electric signals (e.g., TTSignals, wave forms). The first transducer array 70a and the second transducer array 70b, being supplied with the electric signals, causes an electrical current to flow between the first transducer array 70a and the second transducer array 70b. The electrical current generates an electric field (i.e., TTField), having a frequency and an amplitude, to be generated between the first transducer array 70a and the second transducer array 70b.

While the electronic apparatus 50 shown in FIG. 2 comprises only two transducer arrays 70 (i.e., the first transducer array 70a and the second transducer array 70b), in some embodiments, the electronic apparatus 50 may comprise more than two transducer arrays 70.

The electric field generator 54 generates an alternating voltage wave form (i.e., TTSignal) at frequencies in the range from about 50 kHz to about 1 MHz (preferably from about 100 kHz to about 500 kHz). The required voltages are such that an electric field intensity in tissue within the treatment area is in the range of about 0.1 V/cm to about 10 V/cm. To achieve this electric field intensity, the potential difference between the two conductors (e.g., the electrode(s) 100a, 100b, or 100c described below) in each of the first transducer array 70a or the second transducer array 70b is determined by the relative impedances of the system components, e.g., a fraction of the electric field on each component is given by that component's impedance divided by a total circuit impedance.

In certain particular (but non-limiting) embodiments, the first transducer array 70a and the second transducer array 70b generate an alternating electric current and field within a target region of a patient. The target region may comprise at least one tumor (or region including the resection cavity after removal of the tumor), in which case the generation of the alternating electric current and field selectively destroys and/or inhibits growth of the tumor (or cancer cells). The alternating electric current and field may be generated at any frequency suitable for treating a patient, for example that selectively destroys or inhibits growth of the tumor (or cancer cells), such as at any frequency of a TTField.

In certain particular (but non-limiting) embodiments, the alternating electric current and field may be imposed at two or more different frequencies. When two or more frequencies are present, each frequency is selected from any of the above-referenced values, or a range formed from any of the above-referenced values, or a range that combines two integers that fall between two of the above-referenced values.

In order to optimize the electric field (e.g., TTField) distribution, the first transducer array 70a and the second transducer array 70b (pair of transducer arrays 70) may be configured differently depending upon the application in which the pair of transducer arrays 70 are to be used. The pair of transducer arrays 70, as described herein, are externally applied to a patient, that is, are generally applied to the patient's skin, in order to apply the electric current, and electric field (e.g., TTField) thereby generating current within the patient's tissue. Generally, the pair of transducer arrays 70 are placed on the patient's skin by a user such that the electric field is generated across patient tissue within a treatment area. Alternating electric fields that are applied externally can be of a local type or widely distributed type, for example, the treatment of skin tumors and treatment of lesions close to the skin surface.

In one embodiment, the user may be a medical professional, such as a doctor, nurse, therapist, or other person acting under the instruction of a doctor, nurse, or therapist. In another embodiment, the user may be the patient, that is, the patient (and/or a helper) may place the first transducer array 70a and the second transducer array 70b on the patient's treatment area.

According to another exemplary embodiment, the electronic apparatus 50 includes a controller 74. In one embodiment, the controller 74 comprises circuitry configured to control the output of the electric field generator 54, for example, to set the output at the maximal value that does not cause excessive heating of the treatment area. The controller 74 may issue a warning, or the like, when a temperature of the treatment area exceeds a preset limit. A temperature sensor 76 may be mechanically connected to and/or otherwise associated with the first transducer array 70a and/or the second transducer array 70b so as to sense the temperature of the treatment area at either one or both of the first transducer array 70a or the second transducer array 70b as described below in more detail.

In one embodiment, the controller 74 may turn off, or decrease power of the TTSignal generated by the electric field generator 54, if a temperature meets or exceeds a comfortability threshold. In one embodiment, the comfortability threshold is the temperature at which a patient would be made uncomfortable while using the first transducer array 70a and the second transducer array 70b. For example, the comfortability threshold may be a temperature at or about 41 degrees Celsius. In one embodiment, the comfortability threshold is a temperature of between about 39 degrees Celsius and 42 degrees Celsius, or a specific selected temperature between about 39 degrees Celsius and 42 degrees Celsius.

The conductive leads 58 are isolated conductors with a flexible metal shield, preferably grounded thereby preventing spread of any electric field generated by the conductive leads 58. The first transducer array 70a and the second transducer array 70b may have specific shapes and positioning so as to generate the alternating electric field (e.g., TTField) of a desired configuration, direction, and intensity at the treatment area and only at that treatment area so as to focus the treatment.

The specifications of the electronic apparatus 50 as a whole and its individual components are largely influenced by the fact that at the frequency of the alternating electric fields described herein (e.g., TTFields), living systems behave according to their “Ohmic”, rather than their dielectric properties.

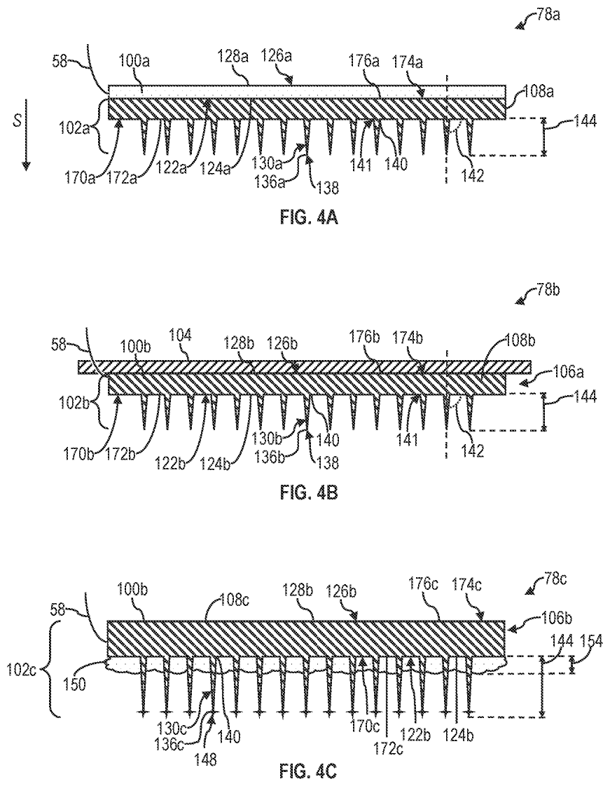

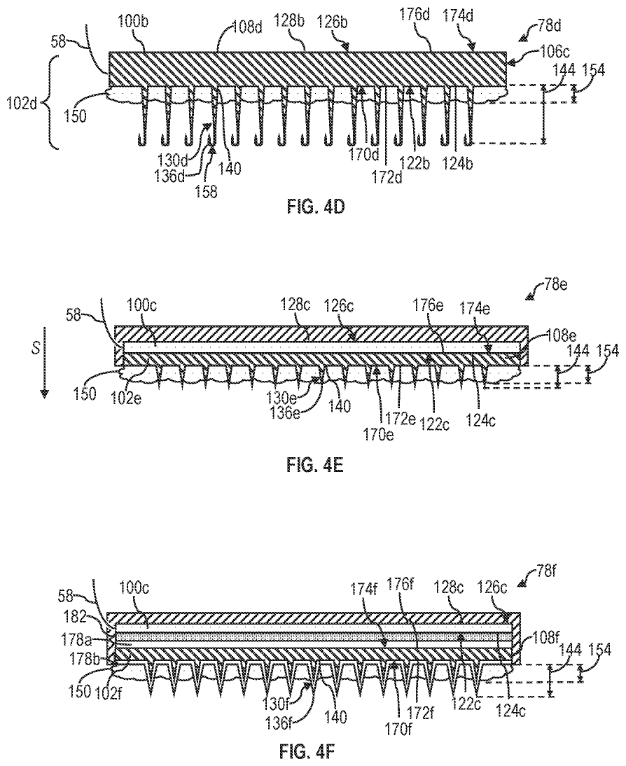

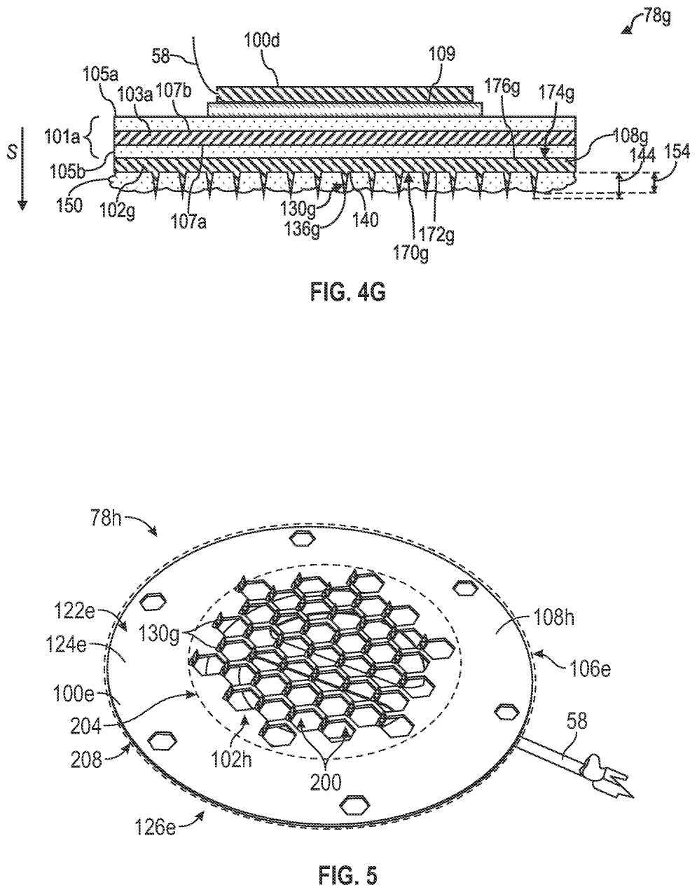

Referring now to FIG. 3, shown therein is a diagram of an exemplary embodiment of the transducer array 70 constructed in accordance with the present disclosure. As shown in FIG. 3, each transducer array 70 is configured as a set of one or more electrode elements 78. In the example shown, the transducer array 70 includes 9 electrode elements 78. Transducer arrays 70 may utilize electrode elements 78 that are capacitively coupled. In the example shown in FIG. 3, the transducer array 70 is configured as multiple electrode elements 78 (for example, about 2 cm in diameter) that are interconnected via flex wires 90 (and connected to the electric field generator 54 via the conductive lead 58). Each electrode element 78 is discussed in more detail below and shown in FIG. 4. In one embodiment, the transducer array 70 includes an outer peripheral edge 84.

Referring now to FIG. 4A, shown therein is a cross-sectional diagram of an exemplary embodiment of a first electrode element 78a constructed in accordance with the present disclosure (cross section through electrode element 78 taken at line 4-4′ in FIG. 3). The first electrode element 78a generally comprises a first electrode 100a operably coupled to the conductive lead 58, and a first transfer member 102a. The first electrode 100a has a first electrode surface 122a on a first skin-facing side 124a, and a second electrode surface 126a on an opposing outwardly-facing second side 128a. The first transfer member 102a has a first substrate surface 170a on a first skin-facing side 172a, and a second substrate surface 174a on an opposing outwardly-facing second side 176a. The first transfer member 102a is located on the first electrode surface 122a on the first skin-facing side 124a of the first electrode 100a and may be electrically connected to the first electrode 100a.

The first transfer member 102a has at least one first protrusion 130a extending in a skin-facing direction, S, the first transfer member 102a is configured to pierce a patient at a target area, e.g., the at least one first protrusion 130a of the first transfer member 102a pierces skin of the patient. The at least one first protrusion 130a includes a base portion 140 and a first end portion 136a.

Generally, a transfer member 102, such as the first transfer member 102a, is an assembly comprising one or more protrusion 130 (hereinafter protrusions 130), e.g., the first protrusion 130a, connected to a support substrate 108, shown in FIG. 4A as a first support substrate 108a. In one embodiment, the first transfer member 102a includes a plurality of the protrusions 130 connected to and extending from the support substrate 108. In some embodiments, the support substrate 108 is solid, while in other embodiments, the support substrate 108 may have one or more void disposed therein (such as shown in FIG. 5). In some embodiments, such as shown in FIGS. 4B-D and FIG. 5, the support substrate 108 is integrated and/or combined with the electrode 100. In one embodiment, the protrusions 130 are microneedles.

The first electrode element 78a is an exemplary embodiment of the electrode element 78 of FIG. 3 and may be used in place of one or more of the electrode element 78 of FIG. 3.

The first electrode 100a comprises and/or consists of at least one conducting element and/or compound, including, by way of example only, elemental silver.

In one embodiment, the first electrode 100a is electrically conductive and comprises, at least in part, a material selected from one or more of the following: silver, gold, tin, aluminum, titanium, platinum, carbon, an alloy thereof, and/or some combination thereof.

In one embodiment, the first transfer member 102a, on the first skin-facing side 124a of the first electrode 100a, comprises one or more first protrusions 130a extending from the first substrate surface 170a of the first transfer member 102a. Each first protrusion 130a may be configured to pierce the patient. By piercing the patient, the first protrusions 130a may restrict or limit movement of the first transfer member 102a, or the first electrode 100a, from the target location of the patient. In other words, by piercing the patient, day-to-day movement of the patient is less likely to dislodge or otherwise move the first electrode 100a (or the transducer array 70) from the target location of the patient. By maintaining the first electrode 100a (and/or the transducer array 70) at the target location, the patient may be confident that the alternating electric field (e.g., TTField) is provided to the same target location between intentional replacement of the transducer arrays 70. The first protrusions 130a are sized to pass through the epidermis layer of the patient's skin. Because electricity is passing through the first protrusions 130a, the first protrusions 130a can enhance the electrical interface between the first electrode 100a and the patient thereby reducing resistance and enhancing the flow of current through the patient.

For example, for the purposes of assessing resistance only, a single patch array (e.g., a transducer array 70 having a single one of the electrode element 78) interfaced to the skin by hydrogel alone (acting as an interface member 150, described below) measured a resistance of 3.24 ohms at 1 kHz; whereas the same total area array interfaced to the same total area of skin such that % of the area was interfaced by hydrogel and % of the area was interfaced by a substrate supporting microneedles measured a resistance of 2.38 ohms at 1 kHz. Even using microneedles (e.g., protrusions 130) to interface to only a small portion of the area is still highly effective at bypassing the high resistance of the keratinized/keratin skin layer and effecting a lower resistance overall.

Each first protrusion 130a may comprise a first end portion 136a and a base portion 140 wherein first protrusion 130a tapers from the base portion 140 towards the first end portion 136a such that the first end portion 136a has a smaller surface area (for example, a cross-sectional area of the protrusion, as described below) than the base portion 140. In some embodiments, the first end portion 136a has a surface area of between 1 μm2 and 50 μm2. In other embodiments, the first end portion 136a has a surface area of between 50 μm2 and 2,000 μm2. In one embodiment, the first end portion 136a has a surface area between ¼ and 1/20 of a surface area of the base portion 140. As used herein, the surface area of the first end portion 136a may be a cross-sectional area of the first protrusion 130a approximately 1 mil (25.4 μm) from a distal tip 138 of the first protrusion 130a (and similarly for the base portion 140, where a cross-sectional area of the base portion 140 is measured at a location of a base 141 of the first protrusion 130a). In one embodiment, the surface area of the first end portion 136a may be selected based on a height 144 (described below). In one embodiment, a diameter of the first end portion 136a is between ¼ and 1/20 of the height 144 of the first protrusion 130a. For example, if the height 144 is 200 μm, then the diameter of the first end portion 136a may be between about 10 μm and 50 μm (and a 10 μm diameter would result in a surface area, cross-sectional area of the first protrusion 130a as described above, of the first end portion 136a being approximately 78.5 μm2, assuming a cone shaped first protrusion 130a).

In some embodiments, the first protrusion 130a is a needle or microneedle (e.g., an elongated cone-shape or a slender rodlike instrument with the first end portion 136a forming a point). In some embodiments, the first protrusion 130a as a needle or microneedle, includes a hollow core extending through the first end portion 136a of the needle or microneedle. In one embodiment, the hollow core may include one or more conductive materials disposed therein. The conductive material may be a salt and may, in some embodiments, be dissolved into an aqueous solution. For example, the hollow core may include a saline solution disposed therein. In another example, the hollow core may include a solution of sodium hyaluronate disposed therein. In some embodiments, the needle or microneedle is constructed of a dielectric material, whereas in other embodiments, the needle or microneedle is constructed of, or coated with, a conductive material.

In one embodiment, each first protrusion 130a has the height 144 between the first end portion 136a and the first substrate surface 170a of the first transfer member 102a. The height 144 may be selected based on the target area of the patient. For example, if the target area of the patient results in placement of the transducer array 70 on the patient's skin, the height 144 of the first protrusion 130a may be selected such that the first end portion 136a of the first protrusion 130a does not reach pain receptors when piercing the patient at the target area, e.g., the height 144 may be selected to be within the range of about 10 μm to 15 μm to avoid the patient's nerves but still pierce through the keratinized/keratin skin layer of the patient's skin, e.g., one or more of the stratum corneum, stratum lucidum, and stratum granulosum layers of the epidermis. In one embodiment, the height 144 may be selected to be within the range of 10 μm to 200 μm such that, when the transducer array 70 is placed on the patient, the first protrusions 130a pierce through the epidermis layer of the patient's skin, but do not pierce so far as to reach the patient's nerves. In one embodiment, the height 144 is preferably between 10 μm and 50 μm.

The first protrusions 130a may be microneedles and may be constructed of metal or ceramic, and may be salt coated. Commercially available microneedles are often constructed of stainless steel. Other microneedles may be made of, for example, silver, gold, platinum, aluminum, tin, titanium, titanium nitride, or iridium oxide. Microneedles could also be configured to be slowly dissolvable, for example, by constructing the microneedles from salts of hyaluronic acid or polylactic acid. In some embodiments, the first protrusions 130a may be positioned on or among dimples such that the extension of the first protrusion 130a is accentuated or modified. The first protrusions 130a may be arranged on a substrate or grid in a regular pattern, such as, for example, the hexagonal grid pattern shown in FIG. 5. In FIG. 5, the grid pattern consists of fused (edge-sharing) regular hexagons with the protrusions present on two opposing edges of the 6 edges for each hexagon. For this and similar types of patterns, shortening the edge lengths of the regular hexagons has the effect of increasing the density of the first protrusions 130a for a given area covered by the grid pattern.

In one embodiment, the first transfer member 102a and/or the first electrode 100a are constructed of a flexible material configured to contour to a shape of the target area, e.g., a location on the patient's body where the transducer array 70 is placed.

In one embodiment, the first transfer member 102a is constructed of a dielectric material. The dielectric material may be any biocompatible material having a dielectric constant of between 10 and 100, or between 30 and 78.5, for example. The dielectric material may further have a rigidity sufficient to pierce the patient. Preferably, the dielectric material is non-conducting to direct current but conducting to alternating current. In one embodiment, the dielectric constant of the first transfer member 102a is selected such that a capacitance of between 100 pF and 1 F is achieved. In one embodiment, the dielectric material comprises a polydimethylsiloxane (PDMS) material. In one embodiment, the dielectric material may comprise one or more of: TiO2, ZrO2, HfO2, ZrTiSn, BaSmTi, SiO2, and the like, or some combination thereof.

In one embodiment, the first transfer member 102a is constructed of a dissolvable material such as a dissolvable material comprising sodium hyaluronate or sodium salt of polylactic acid. The first transfer member 102a, constructed of the dissolvable material, may be constructed such that the first transfer member 102a does not dissolve within 2-3 days and preferably does not dissolve in less than three days, or does not dissolve in less than four days. In some embodiments the first transfer member 102a begins to dissolve after 2, 3, or 4 days. Such embodiments may be easier to remove for cleaning and/or treating the skin before application of a new transducer array 70.

In one embodiment, the first transfer member 102a may further include one or more therapeutic agent within the dissolvable material. In this embodiment, the first transfer member 102a exudes the one or more therapeutic agent as the first transfer member 102a dissolves. The therapeutic agent may be provided in varying concentrations within the dissolvable material such that, as the first transfer member 102a dissolves, a predetermined concentration of the therapeutic agent is released. In this way, a predetermined amount of the therapeutic agent may be released over a three-day period (or other period of time for which the transducer array 70 is attached to the patient). In one embodiment, the therapeutic agent is selected to ameliorate or soothe any adverse events (e.g., dermatitis, lesions or burns) associated with skin irritation. In one embodiment, the therapeutic agent is a chemotherapy agent.

In one embodiment, the first transfer member 102a is constructed of an electrically conductive material. The electrically conductive material may comprise one or more of silver, gold, tin, aluminum, titanium, platinum, stainless steel, carbon, an alloy thereof, and/or some combination thereof. In some embodiments, the first transfer member 102a is exclusive of nickel.

In one embodiment, each first protrusion 130a may extend from the support substrate 108a of the first transfer member 102a at an angle 142 relative to the base portion 140 of the first protrusion 130a. The angle 142 may be between 45° and 135° and is preferably about 90°. In some embodiments, one or more first protrusion 130a may extend from the support substrate 108a of the first transfer member 102a at a first angle relative to the base portion 140 while another of the one or more first protrusion 130a may extend from the support substrate 108 of the first transfer member 102a at a second angle relative to the base portion 140 where the second angle is different from the first angle.

In one embodiment, the first end portion 136a of one or more first protrusion 130a has disposed thereon a salt. The salt may comprise, but is not limited to, one or more of sodium chloride, potassium chloride, and silver nitrate, or a combination thereof. In some embodiments, the first end portion 136a may be constructed of the salt.

Optionally, in this embodiment, and also optionally for other embodiments discussed herein, a layer of dielectric material may be positioned between the electrode (e.g., the first electrode 100a) and the support substrate 108a of the transfer member (e.g., the first transfer member 102a) comprising at least one protrusion 130 (e.g., the first protrusion 130a). In some embodiments, the dielectric material has a dielectric constant of at least 10. The dielectric material may take the form of a ceramic material or a high dielectric polymer as known in the art.

Referring now to FIG. 4B, shown therein is a diagram of an exemplary embodiment of a second electrode element 78b constructed in accordance with the present disclosure. The second electrode element 78b may be constructed similarly to the first electrode element 78a, as described above, with the following exceptions. In some embodiments, and as shown in FIGS. 4B-4D, the transfer member 102 (e.g., 102b, 102c and 102d, respectively) having the support substrate 108 is integrally formed with the first skin-facing side 124 of the second electrode 100b so that a second transfer member 102b and the second electrode 100b is a first unitary structure 106a. In other embodiments, the transfer member 102 can be constructed separately from the electrode 100 (as shown in FIGS. 4A, 4E and 4F, where transfer members 102a, 102e and 102f, respectively, are separate from electrode 100a, 100c and 100c, respectively, in each case). In the latter embodiments, the transfer member 102 and the electrode 100 can be subsequently placed adjacent to each other (as in FIG. 4A and FIG. 4E), or in close proximity to each other (as in FIG. 4F).

In FIG. 4B, the second electrode element 78b comprises the second electrode 100b and the second transfer member 102b combined together in the first unitary structure 106a, the second transfer member 102b formally on the first side 128 of the second electrode 100b. The second transfer member 102b comprises a second support substrate 108b and one or more second protrusion 130b extending from a first surface 170b of support substrate 108b of the second transfer member 102b, the second protrusion 130b having the base portion 140 and a second end portion 136b.

In some embodiments, the second electrode element 78b further comprises a top-coat layer 104. In some embodiments, the second electrode surface 126b of the second electrode 100b is in contact with the top-coat layer 104. In one embodiment, the top-coat layer 104 may increase safety of the transducer array 70 and/or the second electrode element 78b by preventing or limiting contact with the second electrode 100b to guard against accidental electrocution or electrification when the second electrode element 78b is receiving TTSignal(s) and generating an alternating electric field (e.g., TTField). The top-coat layer 104 may be constructed of a durable, non-conductive material, such as a non-conductive fabric. In some embodiments, the non-conductive fabric may have a plurality of perforations. In one embodiment, the top-coat layer 104 has a thickness of less than 1 mm and generally has a thickness of about 0.5 mm.

In one embodiment, the top-coat layer 104 may extend within the outer peripheral edge 84 (FIG. 3) of the transducer array 70 or beyond. In some embodiments, and as shown in FIG. 4B, the top-coat layer 104 extends to cover the second electrode surface 126b of the second electrode 100b. In one embodiment, the top-coat layer 104 may extend beyond any other component of the transducer array 70 and, having an adhesive on a surface of the top-coat layer 104 in contact with the second electrode 100b, adhere to the patient's skin. A top-coat layer 104 may be present in any of the embodiments described herein.

Referring now to FIG. 4C, shown therein is a diagram of an exemplary embodiment of a third electrode element 78c constructed in accordance with the present disclosure. The third electrode element 78c also comprises the second electrode 100b in a second unitary structure 106b with a third transfer member 102c. The third electrode element 78c may be constructed similarly to the second electrode element 78b, as described above, with the following exceptions.

The third transfer member 102c comprises a third support substrate 108c and one or more third protrusion 130c extending from a first substrate surface 170c of the third transfer member 102c, the third protrusion 130c having the base portion 140 and a third end portion 136c. The third end portion 136c may form a barb 148, e.g., a projection in the third end portion 136c, angled away from the third protrusion 130c so as to decrease ease of extraction of the third protrusion 130c from the patient. Such a barb 148 may be present additionally or alternatively on the end portion 136 in any of the embodiments described herein. The third support substrate 108c is constructed of a conductive material that forms both the second electrode 100b and a support substrate.

In some embodiments, and as shown in FIGS. 4C-4G, the electrode element 78 comprises an interface member 150 disposed against the first substrate surface 170 of the support substrate 108c of the transfer member 102 (e.g., the first surface 170c of the third transfer member 102c in FIG. 4C). The interface member 150 (FIGS. 4C-4G) may enhance the electrical interface between the electrode 100 (e.g., the second electrode 100b, the third electrode 100c, the fourth electrode 100d) and the patient's skin. In some embodiments, and as shown in FIG. 4C, the interface member 150 has a height 154 less than the height 144 of the third protrusions 130c, although in other embodiments, the interface member 150 has the height 154 greater than or equal to the height 144 of the third protrusions 130c. For embodiments described herein, the interface member 150 is optional, that is, some embodiments of the electrode element 78 do not include the interface member 150. When present, the interface member 150 may be disposed between the electrode 100 or transfer member 102 and the patient's body in any of the embodiments described herein.

In one embodiment, the interface member 150 is electrically conductive and biocompatible when used for an extended period of time. In one embodiment, the interface member 150 is a gel layer, or a hydrogel layer, constructed in accordance with the gel/hydrogel layers described in U.S. Patent Publication No. 2021/0346693 A1, published Nov. 11, 2021 and entitled “CONDUCTIVE PAD GENERATING TUMOR TREATING FIELD AND METHODS OF PRODUCTION AND USE THEREOF” and U.S. Pat. No. 11,458,298, issued on Oct. 4, 2022, and entitled “ASSEMBLIES CONTAINING TWO CONDUCTIVE GEL COMPOSITIONS AND METHODS OF PRODUCTION AND USE THEREOF”, the entire contents of which are hereby incorporated herein in their entirety.

In one embodiment, the interface member 150 comprises one or more layer of material configured to be one or more of electrically conductive, biocompatible when in contact with the patient's skin for an extended period of time, e.g., from 3 hours to a week at a time, flexible so as to not impede movement of the patient while the transducer array 70 is in place, and resistant to movement on the patient's skin as the patient goes about their daily routine.

In one embodiment, the interface member 150 is constructed of one or more layers of a conductive adhesive layer and, optionally, may include a layer of graphite/anisotropic material. Exemplary embodiments of the interface members 150 may be constructed in accordance with U.S. patent application Ser. No. 17/899,220, filed Aug. 30, 2022 and entitled “ELECTRODE ASSEMBLY WITH A SKIN CONTACT LAYER COMPRISING A CONDUCTIVE ADHESIVE COMPOSITE, AND SYSTEMS AND METHODS OF APPLYING TUMOR TREATING FIELDS USING SAME”, the entire content of which is hereby incorporated herein in its entirety.

In some embodiments, the interface member 150 extends (laterally) as far as the second electrode 100b whereas in other embodiments, the interface member 150 extends at least to the outer peripheral edge 84 of the transducer array 70 or beyond.

Referring now to FIG. 4D, shown therein is a diagram of an exemplary embodiment of a fourth electrode element 78d constructed in accordance with the present disclosure. The fourth electrode element 78d comprises the second electrode 100b and a fourth transfer member 102d (shown as a third unitary structure 106c similar to the first unitary structure 106a in FIG. 4B and the second unitary structure 106b in FIG. 4C). The fourth electrode element 78d may be constructed similarly to the third electrode element 78c, as described above, with the following exceptions. The fourth transfer member 102d comprises a fourth support substrate 108d and one or more fourth protrusion 130d extending from a first surface 170d of the fourth transfer member 102d, the fourth protrusion 130d having the base portion 140 and a fourth end portion 136d. The fourth end portion 136d may form a j-hook 158, e.g., a projection in the fourth end portion 136d, angled away from the fourth protrusion 130d and towards the first substrate surface 170d so as to decrease ease of extraction of the fourth protrusion 130d from the patient.

In some embodiments, as shown in FIG. 4D, the interface member 150 disposed against the first substrate surface 170d of the fourth transfer member 102d of the fourth electrode element 78d has a height 154 less than the height 144 of the fourth protrusion 130d, however, in other embodiments, the interface member 150 has the height 154 greater than or equal to the height 144 of the fourth protrusions 130d.

Referring now to FIG. 4E, shown therein is a cross-section diagram of an exemplary embodiment of a fifth electrode element 78e constructed in accordance with the present disclosure. As shown, the fifth electrode element 78e comprises a third electrode 100c operably coupled to the conductive lead 58, and a fifth transfer member 102e. The third electrode 100c has a first electrode surface 122c on a first skin-facing side 124c and a second electrode surface 126c on an opposing outwardly-facing second side 128c opposite the first skin-facing side 124c. The fifth transfer member 102e comprises a fifth support substrate 108e and has a first substrate surface 170e on a first skin-facing side 172e and a second substrate surface 174e on an opposing outwardly-facing second side 176e opposite the first substrate surface 170e and facing the first skin-facing side 124c of the third electrode 100c.

The third electrode 100c comprises and/or consists of at least one conducting element and/or compound, including, by way of example only, elemental silver. In some embodiments, the third electrode 100c further includes a conductive support layer electrically coupled to the third electrode 100c. The third electrode 100c may be selected from any electrically conductive material having desirable properties such as, but not limited to, high conductivity, strong biocompatibility, and low reactivity with other layers or components of the transducer array 70. In one embodiment, the conductive support layer may be electroplated or otherwise bonded to the third electrode 100c.

In one embodiment, the third electrode 100c is electrically conductive and comprises, at least in part, a material selected from one or more of the following: silver, gold, tin, aluminum, titanium, platinum, carbon, an alloy thereof, and/or some combination thereof.

In one embodiment, the third electrode 100c is constructed of a flexible material configured to contour to a shape of the target area, e.g., a location on the patient's body where the transducer array 70 is placed.

In one embodiment, the fifth transfer member 102e, on the first surface 122a, comprises one or more fifth protrusions 130e extending from a first substrate surface 170e in a skin-facing direction, S. The fifth protrusion 130e may be constructed in accordance with any protrusion 130 described above in more detail with the exception that the fifth protrusion 130e extends from the fifth transfer member 102e which is separate from, i.e., not integrally formed with, the third electrode 100c. Each fifth protrusion 130e may be configured to pierce the patient. Each fifth protrusion 130e may comprise a fifth end portion 136e and a base portion 140 wherein the fifth protrusion 130e may taper from the base portion 140 towards the fifth end portion 136e such that the fifth end portion 136e has a smaller surface area than the base portion 140 as discussed earlier herein. In some embodiments, the fifth protrusion 130e is a needle or microneedle (e.g., an elongated cone-shape or a slender rodlike instrument with the fifth end portion 136e forming a point).

In some embodiments, as shown in FIG. 4E, the interface member 150 is optionally disposed against the first substrate surface 170e of the fifth transfer member 102e and has the height 154 less than height 144 of the fifth protrusion 130e, however, in other embodiments, the interface member 150 has the height 154 greater than or equal to the height 144 of the fifth protrusions 130e.

Referring now to FIG. 4F, shown therein is a cross-section diagram of an exemplary embodiment of a sixth electrode element 78f constructed in accordance with the present disclosure. The sixth electrode element 78f may be constructed in accordance with the fifth electrode element 78e with the exception that the sixth electrode element 78f has a first conductive material 178a disposed on a second substrate surface 174f of a sixth transfer member 102f and a second conductive material 178b disposed on a first substrate surface 170a of the sixth transfer member 102f, and the sixth electrode element 78f optionally includes a dielectric material 182 disposed between the sixth transfer member 102f and the third electrode 100c, e.g., the dielectric material 182 is disposed on a first skin-facing side 124c of the third electrode 100c and on an opposing outwardly-facing second side 176f of the sixth transfer member 102f. In some embodiments, the sixth transfer member 102f comprises a sixth support substrate 108f and/or a dielectric material.

In one embodiment, each of the first conductive material 178a and the second conductive material 178b is electrically conductive and comprises, at least in part, a material selected from one or more of the following: silver, gold, tin, aluminum, titanium, platinum, carbon, an alloy thereof, and/or some combination thereof. In some embodiments, the first conductive material 178a and/or the second conductive material 178b are exclusive of nickel.

In some embodiments, as shown in FIG. 4F, the interface member 150 is optionally disposed against the second conductive material 178b on the first surface 170f of the sixth transfer member 102f and has the height 154 less than height 144 of a sixth protrusion 130f, however, in other embodiments, the interface member 150 has the height 154 greater than or equal to the height 144 of the sixth protrusions 130f. One or more of the sixth protrusions 130f may be as described in any of the embodiments described herein.

Referring now to FIG. 4G, shown therein is a cross-section diagram of an exemplary embodiment of a seventh electrode element 78g constructed in accordance with the present disclosure. The seventh electrode element 78g may be constructed in accordance with the first electrode element 78a with the exception that the seventh electrode element 78g has a conductive layer comprising a layer of conductive anisotropic material 103a positioned between the fourth electrode 100d and a seventh transfer member 102g (FIG. 4G). To provide an improved contact between the layer of conductive anisotropic material 103a and adjacent components of the seventh electrode element 78g, optionally (and as shown in FIG. 4G), the layer of conductive anisotropic material 103a may have one or more surrounding layer 105 of either conductive gel (such as, for example, hydrogel) or a conductive adhesive on one or both surfaces (e.g., a first surface 107a and a second surface 107b) of the layer of conductive anisotropic material 103a, shown in FIG. 4G as a first surrounding layer 105a and a second surrounding layer 105b. This 3-layer construct is also shown in FIG. 4G as a conductive layer 101a. In this case, the one or more surrounding layers 105 on either side of the conductive anisotropic material 103a may be the same or different, e.g., constructed of the same or different material.

In some embodiments, the seventh electrode element 78g may further comprise a dielectric material 109 positioned between the fourth electrode 100d and the seventh transfer member 102g comprising at least one seventh protrusion 130g. In some embodiments (and as shown in FIG. 4G), the conductive layer 101a of the conductive anisotropic material 103a may be positioned between the dielectric material 109 and the seventh transfer member 102g. In one embodiment, the dielectric material 109 may be constructed in accordance with the dielectric material 182.

In some embodiments, the conductive anisotropic material 103a may comprise a sheet of graphite. In some embodiments, the conductive anisotropic material 103a may comprise a sheet of pyrolytic graphite. In some embodiments, the conductive anisotropic material 103a may comprise a graphite foil made from compressed high purity exfoliated mineral graphite, or graphitized polymer film. In some embodiments, the conductive anisotropic material 103a may be nonmetallic.

An example of pyrolytic graphite is Pyrolytic Graphite Sheet (PGS), available from Panasonic Industry, Kadoma, Osaka, Japan. An example of graphite foil made from compressed high purity exfoliated mineral graphite is MinGraph® 2010A Flexible Graphite, available from Mineral Seal Corp., Tucson, Arizona, USA. An example of graphitized polymer film, e.g., graphitized polyimide film, is supplied by Kaneka Corp., Moka, Tochigi, Japan. In alternative embodiments, conductive anisotropic materials 103a other than graphite may be used instead of graphite.

In one embodiment, the conductive anisotropic material 103a has surface area, e.g., an area of the first surface 107a. The surface area of the conductive anisotropic material 103a may cover one or more electrode element 78 within the transducer array 70 and may include additional surface area, for example, to cover the seventh transfer member 102g, according to embodiments disclosed herein. The conductive anisotropic material 103a may be electrically connected to the electrode (or, optionally, to the dielectric layer) via a layer of hydrogel or conductive adhesive and may be connected to the seventh transfer member 102g via a layer of hydrogel or conductive adhesive, such as shown in FIG. 4G as the first surrounding layer 105a and the second surrounding layer 105b. In one embodiment, the first surrounding layer 105a and the second surrounding layer 105b may be constructed in accordance with the interface member 150 described above.

In some embodiments, the conductive anisotropic material 103a may have a first resistance in a direction that is perpendicular to the first surface 107a, and a second resistance of the conductive anisotropic material 103a in directions that are parallel to the first surface 107a may be, for example, less than half (or even less than one tenth, or less than one hundredth) of the first resistance. Accordingly, current may be spread across the surface area of the conductive anisotropic material 103a and pass to the patient's skin via the seventh transfer member 102g across the whole area, e.g., first surface 107a, of the conductive anisotropic material 103a (see, for example, United States Patent Publications 2023/0037806, published Feb. 9, 2023, and 2023/0065587, published Mar. 2, 2023). The conductive layer 101a of the conductive anisotropic material 103a may be included in any of the embodiments disclosed herein, optionally with one or more surrounding layer 105 of either a conductive gel (hydrogel) or a conductive adhesive, on one or both sides 107 of the conductive layer 101a of the conductive anisotropic material 103a.

Referring now to FIG. 5, shown therein is a perspective view of an exemplary embodiment of an eighth electrode element 78h constructed in accordance with the present disclosure. The eighth electrode element 78h comprises a fifth electrode 100e (in combination with an eighth transfer member 102h in a fifth unitary structure 106e) comprising an eighth support substrate 108h and having a plurality of eighth protrusions 130h. One or more of the eighth protrusions 130h may be constructed in accordance with any embodiment of the protrusion 130 described herein.

In one embodiment, the eighth transfer member 102h of the eighth electrode element 78h comprises forty-two (42) eighth protrusions 130h. In some embodiments, the eighth transfer member 102h comprises between 4 of the eighth protrusions 130h and 1,000 of the eighth protrusions 130h. In some embodiments, the eighth transfer member 102h has a protrusion density of between 4 and 1,000 protrusions per cm2 on a first skin-facing side 124e of the fifth electrode 100e. In one embodiment, the protrusion density may be selected based on a desirable total contact surface area of the transducer array 70. For example, each eighth protrusion 130h, having a height and a base surface area, has a contact surface area. If a particular total contact surface area is selected for each transducer array 70, the protrusion density may be increased such that the total contact surface area of all eighth protrusions 130h, and a surface area of any portion of the first skin-facing side 124e without a protrusion 130, e.g., the eighth protrusion 130h, meets or approaches the particular total contact surface area.

In one embodiment, the eighth transfer member 102h comprises a plurality of voids 200 extending from a first surface 122e of the first skin-facing side 124e to a second surface 126e of an opposing outwardly-facing second side 128e of the fifth electrode 100e. As shown, the plurality of voids 200 may be disposed between each of the plurality of eighth protrusions 130h. The plurality of voids 200 may be sized such that, when the interface member 150 is present and is constructed of a gel, hydrogel or conductive adhesive layer as described above, the gel, hydrogel or conductive adhesive may pass through one of more of the plurality of voids 200. By including the gel (or hydrogel or conductive adhesive) as the interface member 150 passing through one or more of the plurality of voids 200, adhesive properties of the gel (or hydrogel or conductive adhesive) may further assist maintaining the eighth electrode element 78h at the target area of the patient. Such voids 200 as described for the embodiment of FIG. 5 may also be present in other embodiments described herein, such as, for example, the embodiments of FIGS. 4A-4G.

In one embodiment, as shown in FIG. 5, the eighth transfer member 102h comprises an inner portion 204 and an outer portion 208. In this embodiment, the plurality of eighth protrusions 130h may be evenly disposed within the inner portion 204 while no eighth protrusions 130h are disposed in the outer portion 208. In other embodiments, the plurality of eighth protrusions 130h may be evenly disposed in the outer portion 208 while no eighth protrusions 130h are disposed in the inner portion 204; or may be evenly disposed along the entire surface of the eighth transfer member 102h. In some embodiments, a plurality of eighth protrusions 130h may be evenly disposed in the inner portion 204, and a plurality of eighth protrusions 130h may be evenly disposed in the outer portion 208, wherein the amount or density of the eighth protrusions 130h differs between those in the inner portion 204 and those in the outer portion 208. Further, an uneven distribution of eighth protrusions 130h may exist in either the inner portion 204 or the outer portion 208, or both. In some embodiments, the plurality of voids 200 may be evenly disposed in the inner portion 204, the outer portion 208, or both the inner portion 204 and the outer portion 208. Disposition of the plurality of voids 200 may be made irrespective of the disposition of the plurality of eighth protrusions 130h or may correspond to the disposition of the plurality of eighth protrusions 130h.

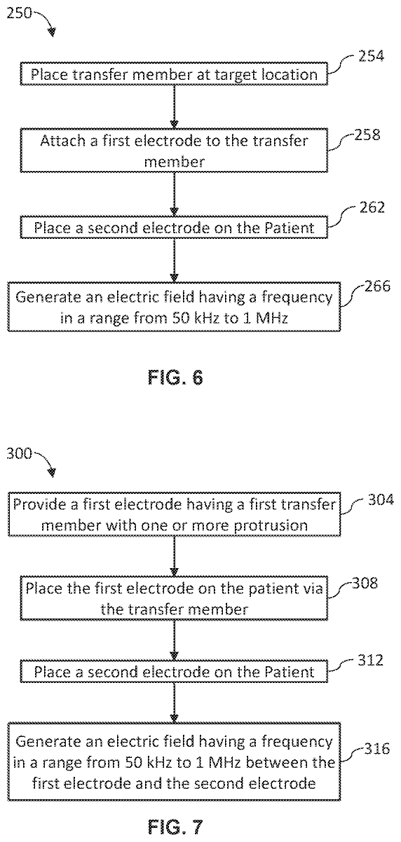

Referring now to FIG. 6, shown therein is an exemplary embodiment of a process 250 of using the electronic apparatus 50 (FIG. 2) and the transducer array 70 (FIG. 3) to apply an alternating electric field (e.g., TTField) to a patient in accordance with the present disclosure. The process 250 generally comprises the steps of: placing a transfer member at a target location (step 254); attaching a first electrode to the transfer member (step 258); placing a second electrode on the patient (step 262); and generating an alternating electric field having a frequency in a range of from about 50 kHz to about 1 MHz (step 266). The steps need not be performed in the order presented here.

In one embodiment, placing a transfer member at a target location (step 254) may be performed by the user. In one embodiment, before applying the transfer member 102 to the target location (e.g., the skin of the patient), the patient's skin may need to be cleaned (e.g., such as, but not limited to, cleansing of the skin of foreign matter or biological matter, and/or shaving of the skin, if necessary) to enable the transfer member 102 to adhere to the patient. In some embodiments, the protrusions 130 of the transfer member 102 may have a height great enough to extend through foreign or biological matter on the patient's skin and pierce the patient. In some embodiments, the height of the protrusions 130 is configured so as to allow the protrusions 130 to extend into or through the epidermis layer of the patient's skin.

In one embodiment, placing a transfer member at a target location (step 254) may include placing an interface member 150 at the target location prior to placing the transfer member 102 on the interface member 150; or placing an interface member 150 on the transfer member 102 prior to placing the transfer member 102 at the target location. In some embodiments, the interface member 150 may be a gel, such as a hydrogel, or a conductive adhesive layer and/or material.

In one embodiment, attaching a first electrode (such as one of the first electrode 100a, the second electrode 100b, the third electrode 100c, and the fourth electrode 100d) to the transfer member 102 (step 258) may include attaching the first electrode to the transfer member 102 which has been placed at the target location. Attaching the first electrode to the transfer member 102 may be performed by the user, or by someone under the instruction of the user or a medical professional.

In one embodiment, attaching the first electrode 100 to the transfer member 102 (step 258) may include attaching the first electrode 100 to the transfer member 102 prior to placing the transfer member 102 at the target location. In this embodiment, the first two steps (i.e., the step 254 and the step 258) may be combined into one step: attaching the first electrode at the target location.

For example, in one embodiment, the second electrode 100b and the second transfer member 102b are a unitary structure 106, as described earlier herein (e.g., FIGS. 4B-D, FIG. 5). In this embodiment, the first two steps (i.e., the step 254 and the step 258) may be combined into one step: placing the first electrode having a transfer member at the target location.

In one embodiment, placing a second electrode (such as one of the first electrode 100a, the second electrode 100b, the third electrode 100c, and the fourth electrode 100d) on the patient (step 262) includes placing the second electrode at a second target location. In some embodiments, placing the second electrode on the patient may include placing the second transducer array 70b on the patient.

In one embodiment, placing the second electrode on the patient (step 262) may be performed by the user, or a person under the instruction of the user or a medical professional. In one embodiment, before placing the second electrode on the patient, the patient may need to be cleaned (e.g., such as, but not limited to, cleansing of the skin of foreign matter or biological matter, and/or shaving of the skin, if necessary) to enable the second electrode to adhere to the patient.

The step of generating an alternating electric field (TTField) (step 266) may be performed by the electric field generator 54 and may be instantiated by an operation performed by the user, a person under the direction of the user or a medical professional, or the controller 74. In one embodiment, step 266 may be performed more than one time and the period of time for which the step 266 is performed a first time may be the same as or different from the period of time for which the step 266 is performed a second time (or other period(s) of time beyond the second time).

In some embodiments, step 266 is only performed once before the process 250 is repeated. There may be a time period between each time the process 250 is repeated. Each time the process 250 is repeated, the time period may be the same as or different from the previous time period. Each time the process 250 is repeated, the first electrode and the second electrode may be placed in the same or a different target location.

The step of generating an alternating electric field (TTField) (step 266) may be performed by generating the alternating electric current and field at two or more different frequencies within the range of 50 kHz to 1 MHz. When two or more frequencies are present, each frequency is selected from any of the above-referenced values, or a range formed from any of the above-referenced values, or a range that combines two integers that fall within the range of the above-referenced values.

In one embodiment, the step of generating an alternating electric field (TTField) (step 266) may be performed by supplying a first alternating electric current and field to a first pair of electrodes 100 (e.g., two or more of the first electrode 100a, the second electrode 100b, the third electrode 100c, and the fourth electrode 100d) for a first period of time and supplying a second alternating electric current and field to a second pair of electrodes 100 for a second period of time. In one embodiment, the first period of time may be of the same or a similar duration to the second period of time whereas in other embodiments, the first period of time may be of a different duration to the second period of time. Additionally, the first period of time may or may not overlap with the second period of time.

Referring now to FIG. 7, shown therein is a process flow diagram of an exemplary embodiment of a second process 300 of using the electronic apparatus 50 and the transducer array 70 in accordance with the present disclosure. The second process 300 generally comprises: providing a first electrode having a first transfer member with one or more protrusion (step 304); placing the first electrode on the patient via the transfer member (step 308); placing a second electrode on the patient (step 312); and generating an electric field having a frequency in a range from 50 kHz to 1 MHz between the first electrode and the second electrode (step 316).

In one embodiment, providing a first electrode having a first transfer member with one or more protrusion (step 304) may include affixing the first transfer member 102 to the first electrode 100 wherein the first transfer member 102 comprises one or more protrusion 130 (such as protrusions 130a-130g).

In one embodiment, providing a first electrode having a first transfer member with one or more protrusion (step 304) may include the first electrode 100 having the first transfer member 102 wherein the first electrode 100 and the first transfer member 102 are a unitary structure 106 (as shown in FIGS. 4B-D, FIG. 5).

In one embodiment, placing the first electrode on the patient via the transfer member (step 308) includes placing the first electrode 100 on the patient via the transfer member 102 such that the plurality of protrusions 130 extend into or through an epidermis and/or keratinized skin layer of the patient.

In one embodiment, placing a second electrode on the patient (step 312) includes placing the second electrode having a second transfer member with one or more protrusion 130 on the patient. In some embodiments, placing the second electrode on the patient (step 312) further include placing the second electrode having the second transfer member on the patient, wherein the second electrode and the second transfer member are a unitary structure 106 (such as in FIGS. 4B-D, FIG. 5).

In one embodiment, placing a second electrode on the patient (step 312) includes placing the second electrode on the patient wherein an interface member 150 is disposed at least partially between the patient and the second electrode.

In one embodiment, placing a second electrode on the patient (step 312) includes placing the second electrode on the patient via the second transfer member such that the plurality of protrusions extend into or through an epidermis and/or keratinized skin layer of the patient.

In one embodiment, generating an electric field having a frequency in a range from 50 kHz to 1 MHz between the first electrode and the second electrode (step 316) includes supplying a first alternating electric current and field to a first pair of electrodes 100 (e.g., two or more of the first electrode 100a, the second electrode 100b, the third electrode 100c, and the fourth electrode 100d) for a first period of time and supplying a second alternating electric current and field to a second pair of electrodes 100 for a second period of time. In one embodiment, the first period of time may be of the same or a similar duration to the second period of time whereas in other embodiments, the first period of time may be of a different duration to the second period of time. Additionally, the first period of time may or may not overlap with the second period of time.

In one embodiment, generating an electric field having a frequency in a range from 50 kHz to 1 MHz between the first electrode and the second electrode (step 316) may be performed in accordance with step 226 described above.

In an exemplary embodiment of another process of using the electronic apparatus 50 and the transducer array 70 to apply an alternating electric field to a patient in accordance with the present disclosure, the process generally comprises the steps of: placing a first electrode on the patient; placing a second electrode on the patient; and generating an alternating electric field having a frequency in a range of from about 50 kHz to about 1 MHz between the electrodes, wherein one or both of the first and second electrodes include a transfer member 102 and a plurality of protrusions 130 extending from the transfer member 102, and wherein the first and/or second electrodes are placed on the patient such that the plurality of protrusions 130 extend into or through an epidermis of the patient.

### Illustrative Embodiments

The following is a number list of non-limiting illustrative embodiments of the inventive concept disclosed herein:

Illustrative Embodiment 1. A transducer array, comprising:
an electrode having a first skin-facing side and an opposing outwardly-facing second side, the electrode comprising a first electrode surface on the first skin-facing side, and a second electrode surface on the opposing outwardly-facing second side; and
a transfer member comprising a support substrate having a first substrate surface on a first skin-facing side, wherein the support substrate is located on the first skin-facing side of the electrode and wherein the transfer member further comprises at least one protrusion extending in a skin-facing direction from the first substrate surface and is configured to pierce a patient at a target area, the at least one protrusion having a base portion and an end portion.

Illustrative Embodiment 2. The transducer array of Illustrative Embodiment 1, wherein the transfer member is disposed on the first electrode surface of the electrode.

Illustrative Embodiment 3. The transducer array of Illustrative Embodiment 1, wherein the at least one protrusion has a height extending from the base portion to the end portion, the at least one protrusion configured to pierce a keratin skin layer of the patient at the target area.

Illustrative Embodiment 4. The transducer array of Illustrative Embodiment 3, wherein the height of the at least one protrusion is in a range of 10 μm to 200 μm.

Illustrative Embodiment 5. The transducer array of Illustrative Embodiment 1, wherein the transfer member is constructed of a flexible material configured to contour to a shape of the target area.

Illustrative Embodiment 6. The transducer array of Illustrative Embodiment 1, wherein the transfer member is constructed of a dielectric material.

Illustrative Embodiment 7. The transducer array of Illustrative Embodiment 6, wherein the dielectric material has a dielectric constant between 10 and 100.

Illustrative Embodiment 8. The transducer array of Illustrative Embodiment 1, wherein the transfer member is constructed of a material comprising sodium hyaluronate.

Illustrative Embodiment 9. The transducer array of Illustrative Embodiment 1, wherein the transfer member is constructed of a conductive material.

Illustrative Embodiment 10. The transducer array of Illustrative Embodiment 9, wherein the conductive material is exclusive of nickel.

Illustrative Embodiment 11. The transducer array of Illustrative Embodiment 9, wherein the conductive material comprises titanium.

Illustrative Embodiment 12. The transducer array of Illustrative Embodiment 9, wherein the conductive material comprises stainless steel.

Illustrative Embodiment 13. The transducer array of Illustrative Embodiment 9, wherein the conductive material comprises one or more of silver, gold, carbon, platinum, titanium, titanium nitride, iridium oxide, stainless steel, and combinations thereof.

Illustrative Embodiment 14. The transducer array of Illustrative Embodiment 1, wherein the at least one protrusion includes one or more of a needle and a microneedle, wherein the needle or microneedle has an elongated cone-shape or is a slender rodlike instrument with the end portion forming a point and having a height selected to pierce through an epidermis layer of the patient's skin, but to not pierce so far as to reach the patient's nerves.

Illustrative Embodiment 15. The transducer array of Illustrative Embodiment 1, wherein the support substrate is the electrode whereby the electrode and the transfer member are a unitary structure.

Illustrative Embodiment 16. The transducer array of Illustrative Embodiment 1, wherein the transfer member comprises a dielectric material, the transfer member having a first conductive material disposed on a first surface and a second conductive material disposed on a second surface, the first conductive material and the second conductive material selected from silver, gold, carbon, platinum, titanium, titanium nitride, iridium oxide, stainless steel, and combinations thereof.

Illustrative Embodiment 17. The transducer array of Illustrative Embodiment 1, further comprising a dielectric material positioned between a first skin-facing side of the electrode and a first substrate surface of the transfer member.

Illustrative Embodiment 18. The transducer array of Illustrative Embodiment 1, wherein the transfer member has a protrusion density of between 4 protrusions per cm2 and 1,000 protrusions per cm2.

Illustrative Embodiment 19. The transducer array of Illustrative Embodiment 1, wherein the transfer member further comprises a plurality of voids extending from a second substrate surface to the first substrate surface.

Illustrative Embodiment 20. The transducer array of Illustrative Embodiment 19, wherein one or more of the plurality of voids is sized such that one or more of a gel, a hydrogel, and a conductive adhesive can pass through the one or more of the plurality of voids.

Illustrative Embodiment 21. The transducer array of Illustrative Embodiment 1, wherein the end portion of the at least one protrusion is constructed of, or embedded with, a salt.

Illustrative Embodiment 22. The transducer array of Illustrative Embodiment 21, wherein the salt comprises one or more of sodium chloride, potassium chloride, sodium hyaluronate, and silver nitrate, or a combination thereof.

Illustrative Embodiment 23. The transducer array of Illustrative Embodiment 1, wherein the at least one protrusion includes a plurality of protrusions extending from and disposed on the transfer member.

Illustrative Embodiment 24. The transducer array of Illustrative Embodiment 23, wherein the support substrate has a second substrate surface, and wherein the support substrate further comprises a plurality of voids extending from the second substrate surface to the first substrate surface, the plurality of voids disposed between pairs of the plurality of protrusions.

Illustrative Embodiment 25. The transducer array of Illustrative Embodiment 23, wherein the plurality of protrusions are disposed evenly on the support substrate.

Illustrative Embodiment 26. The transducer array of Illustrative Embodiment 23, wherein the support substrate further comprises an inner portion and an outer portion, and wherein the plurality of protrusions is disposed within either the inner portion or the outer portion of the support substrate.

Illustrative Embodiment 27. The transducer array of Illustrative Embodiment 26, wherein the plurality of protrusions are disposed evenly within either the inner portion or the outer portion of the support substrate.

Illustrative Embodiment 28. The transducer array of Illustrative Embodiment 23, wherein the support substrate further comprises an inner portion and an outer portion, wherein the plurality of protrusions is disposed within both the inner portion and the outer portion of the support substrate, and wherein a first density of the plurality of protrusions disposed within the inner portion of the support substrate differs from a second density of the plurality of protrusions disposed within the outer portion of the support substrate.

Illustrative Embodiment 29. The transducer array of Illustrative Embodiment 1, wherein the at least one protrusion extending from the support substrate extends at an angle between 45 degrees and 135 degrees relative to the first substrate surface supporting the base portion.

Illustrative Embodiment 30. The transducer array of Illustrative Embodiment 29, wherein the at least one protrusion extending from the support substrate extends at an angle of 90 degrees relative to the first substrate surface supporting the base portion.

Illustrative Embodiment 31. The transducer array of Illustrative Embodiment 1, wherein the at least one protrusion extending from the support substrate includes a first protrusion and a second protrusion, and wherein the first protrusion extends at a first angle relative to the electrode and the second protrusion extends at a second angle relative to the electrode, and wherein the first angle is different from the second angle.

Illustrative Embodiment 32. The transducer array of Illustrative Embodiment 1, wherein the at least one protrusion extending from the transfer member comprises one or more of silver, gold, carbon, platinum, titanium, titanium nitride, iridium oxide, and stainless steel, or a combination thereof.

Illustrative Embodiment 33. The transducer array of Illustrative Embodiment 1, wherein the protrusion has a cross-sectional area of between 1 μm2 and 50 μm2 within 1 mil from the end portion.

Illustrative Embodiment 34. The transducer array of Illustrative Embodiment 33, wherein the protrusion is a needle.

Illustrative Embodiment 35. The transducer array of Illustrative Embodiment 33, wherein the protrusion is a microneedle.

Illustrative Embodiment 36. The transducer array of Illustrative Embodiment 1, further comprising an interface member disposed against the first support surface of the support substrate.

Illustrative Embodiment 37. The transducer array of Illustrative Embodiment 36, wherein the interface member is one or more of a gel, a hydrogel, and a conductive adhesive layer.

Illustrative Embodiment 38. The transducer array of Illustrative Embodiment 37, wherein the interface member has a first height less than a second height of the at least one protrusion.

Illustrative Embodiment 39. The transducer array of Illustrative Embodiment 1, further comprising an interface member disposed between the first electrode surface of the electrode and the second substrate surface of the support substrate, wherein the interface member is one or more of a gel, a hydrogel, and a conductive adhesive layer.

Illustrative Embodiment 40. The transducer array of Illustrative Embodiment 1, further comprising a conductive material and wherein the at least one protrusion is either a needle or microneedle having a hollow core, the hollow core having the conductive material disposed therein.

Illustrative Embodiment 41. The transducer array of Illustrative Embodiment 40, wherein the needle or microneedle comprises a dielectric material.

Illustrative Embodiment 42. The transducer array of Illustrative Embodiment 40, wherein the conductive material is a salt.

Illustrative Embodiment 43. The transducer array of Illustrative Embodiment 42, wherein the salt is dissolved in an aqueous solution.

Illustrative Embodiment 44. The transducer array of Illustrative Embodiment 1, wherein the at least one protrusion further includes a barb extending from or formed by the end portion.

Illustrative Embodiment 45. The transducer array of Illustrative Embodiment 1, wherein the at least one protrusion further includes a J-hook extending from or formed by the end portion.

Illustrative Embodiment 46. The transducer array of Illustrative Embodiment 1, further comprising a top-coat layer disposed on the second electrode surface of the electrode.

Illustrative Embodiment 47. The transducer array of Illustrative Embodiment 46, wherein the top-coat layer is nonconductive.

Illustrative Embodiment 48. The transducer array of Illustrative Embodiment 46, wherein the top-coat layer comprises a first surface and a second surface and wherein the first surface is in contact with the second surface of the electrode and extends beyond the second surface of the electrode.

Illustrative Embodiment 49. The transducer array of Illustrative Embodiment 46, wherein the top-coat layer comprises a first surface and a second surface, the first surface having an adhesive affixed thereto, and wherein the first surface of the top-coat layer is in contact with the second electrode surface of the electrode.

Illustrative Embodiment 50. A method, comprising:
placing a transfer member at a target location on a patient such that at least one microneedle of the transfer member pierces skin of the patient, the at least one microneedle having a base portion and an end portion, a first cross-sectional area of the end portion being less than a second cross-sectional area of the base portion;
attaching a first electrode to the transfer member at the target location;
placing a second electrode on the patient; and
activating an electric field generator to provide an alternating current waveform for a period of time to the first electrode and the second electrode, the alternating current waveform having a frequency in a range between 50 kHz-1 MHz.

Illustrative Embodiment 51. The method of Illustrative Embodiment 50, further comprising: placing an interface member on the target location prior to placing the transfer member at the target location on the patient.

Illustrative Embodiment 52. The method of Illustrative Embodiment 50, further comprising: placing an interface member on the transfer member prior to placing the transfer member at the target location on the patient.

Illustrative Embodiment 53. The method of Illustrative Embodiment 50, wherein the transfer member is constructed of an electrically conductive material and wherein the method further comprises passing the alternating current waveform through the transfer member and into the patient.

Illustrative Embodiment 54. A method, comprising:
placing a transfer member at a target location on a patient such that at least one microneedle of the transfer member pierces skin of the patient;
interfacing an electrode with the patient; and
passing a tumor treating field through the electrode and the at least one microneedle of the transfer member, the tumor treating field having a frequency in a range between 50 kHz-1 MHz.

Illustrative Embodiment 55. A method, comprising:
placing a first electrode on a patient;
placing a second electrode on the patient;
generating an alternating electric field having a frequency in a range of from about 50 kHz to about 1 MHz between the first electrode and the second electrode; and
wherein one or both of the first electrode and the second electrode includes a transfer member having a plurality of microneedles extending from the transfer member, and wherein one or more of the first electrode and the second electrode includes the transfer member and is placed on the patient such that the plurality of microneedles extend into or through an epidermis of the patient.

Illustrative Embodiment 56. A method, comprising:
placing a unitary structure comprising a first electrode and a transfer member at a target location on a patient, such that at least one microneedle of the transfer member pierces skin of the patient, the at least one microneedle having a base portion and an end portion, a first cross-sectional area of the end portion being less than a second cross-sectional area of the base portion;
placing a second electrode on the patient; and
activating an electric field generator to provide an alternating current waveform for a period of time to the first electrode and the second electrode, the alternating current waveform having a frequency in a range between 50 kHz-1 MHz.

Illustrative Embodiment 57. A transducer array, comprising:
an electrode having a first electrode surface on a first skin-facing side, and a second electrode surface on an opposing outwardly-facing second side;
a conductive anisotropic material having a first side and a second side, the second side of the conductive anisotropic material disposed on the first skin-facing side of the electrode; and
a transfer member having a first surface on a first skin-facing side, and a second surface on an opposing outwardly-facing second side, wherein the transfer member is disposed adjacent the first side of the conductive anisotropic material, the transfer member having at least one protrusion extending in a skin-facing direction from the first surface of the transfer member and is configured to pierce a patient at a target area, the at least one protrusion having a base portion and an end portion.

Illustrative Embodiment 58. The transducer array of Illustrative Embodiment 57, further comprising a dielectric material disposed between the electrode and the conductive anisotropic material.

Illustrative Embodiment 59. The transducer array of Illustrative Embodiment 58, wherein the dielectric material has a dielectric constant of between 10 and 100.

Illustrative Embodiment 60. The transducer array of Illustrative Embodiment 57, further comprising at least one of a first surrounding layer disposed between the electrode and the conductive anisotropic material and a second surrounding layer disposed between the conductive anisotropic material and the transfer member, wherein at least one of the first surrounding layer and the second surrounding layer is electrically conductive.

Illustrative Embodiment 61. The transducer array of Illustrative Embodiment 60, wherein at least one of the first surrounding layer and the second surrounding layer is constructed of one or more of a conductive hydrogel and a conductive adhesive.

Illustrative Embodiment 62. The transducer array of Illustrative Embodiment 60, wherein the first surrounding layer and the second surrounding layer is operable to electrically couple the electrode and the conductive anisotropic material.

Illustrative Embodiment 63. An electrode element, comprising:
a transfer member having a support substrate constructed of a conductive material and a plurality of protrusions extending from a surface of the support substrate, the plurality of protrusions being microneedles,
an electrode being the support substrate of the transfer member whereby the transfer member and the electrode are a unitary structure; and
an interface element positioned between the microneedles.

Illustrative Embodiment 64. The electrode element of Illustrative Embodiment 63, wherein the interface element is at least one of a conductive adhesive and a hydrogel.

Illustrative Embodiment 65. A transducer array, comprising:
an electrode having a first skin-facing side and an opposing outwardly-facing second side, the electrode comprising a first electrode surface on the first skin-facing side, and a second electrode surface on the opposing outwardly-facing second side; and
a transfer member comprising a support substrate having a first substrate surface on a first skin-facing side, wherein the support substrate is located on the first skin-facing side of the electrode and wherein the transfer member further comprises at least one protrusion extending in a skin-facing direction from the first substrate surface and is configured to pierce a patient at a target area, the at least one protrusion having a base portion and an end portion.

Illustrative Embodiment 66. The transducer array of illustrative embodiment 65, wherein the transfer member is constructed of a flexible material configured to contour to a shape of the target area.

Illustrative Embodiment 67. The transducer array of illustrative embodiment 65, wherein the transfer member is constructed of a conductive material.

Illustrative Embodiment 68. The transducer array of illustrative embodiment 65, wherein the at least one protrusion includes one or more of a needle and a microneedle, wherein the needle or microneedle has an elongated cone-shape or is a slender rodlike instrument with the end portion forming a point and having a height selected to pierce through an epidermis layer of a patient's skin, but to not pierce so far as to reach the patient's nerves.

Illustrative Embodiment 69. The transducer array of illustrative embodiment 65, wherein the support substrate is the electrode whereby the electrode and the transfer member are a unitary structure.

Illustrative Embodiment 70. The transducer array of illustrative embodiment 65, further comprising a dielectric material positioned between a first skin-facing side of the electrode and the first substrate surface of the transfer member.

Illustrative Embodiment 71. The transducer array of illustrative embodiment 65, wherein the support substrate comprises a second substrate surface opposite to the first substrate surface, and wherein the transfer member further comprises a plurality of voids extending from the second substrate surface to the first substrate surface.

Illustrative Embodiment 72. The transducer array of illustrative embodiment 71, wherein one or more of the plurality of voids is sized such that one or more of a gel, a hydrogel, and a conductive adhesive can pass through the one or more of the plurality of voids.

Illustrative Embodiment 73. The transducer array of illustrative embodiment 65, wherein the end portion of the at least one protrusion is constructed of, or is embedded with, a salt.

Illustrative Embodiment 74. The transducer array of illustrative embodiment 65, wherein the at least one protrusion further comprises a plurality of protrusions, and wherein the support substrate further comprises a second substrate surface, and wherein the transfer member further comprises a plurality of voids extending from the second substrate surface to the first substrate surface, the plurality of voids disposed between pairs of the plurality of protrusions.

Illustrative Embodiment 75. The transducer array of illustrative embodiment 74, wherein the support substrate further comprises an inner portion and an outer portion, and wherein the plurality of protrusions is disposed within either the inner portion or the outer portion of the support substrate.

Illustrative Embodiment 76. The transducer array of illustrative embodiment 74, wherein the support substrate further comprises an inner portion and an outer portion, wherein the plurality of protrusions is disposed within both the inner portion and the outer portion of the support substrate, and wherein a first density of the plurality of protrusions disposed within the inner portion of the transfer member differs from a second density of the plurality of protrusions disposed within the outer portion of the support substrate.

Illustrative Embodiment 77. The transducer array of illustrative embodiment 65, wherein the at least one protrusion extending from the support substrate includes a first protrusion and a second protrusion, and wherein the first protrusion extends at a first angle relative to the electrode and the second protrusion extends at a second angle relative to the electrode, and wherein the first angle is different from the second angle.

Illustrative Embodiment 78. The transducer array of illustrative embodiment 65, further comprising an interface member disposed against the first substrate surface of the support substrate.

Illustrative Embodiment 79. The transducer array of illustrative embodiment 78, wherein the interface member is one or more of a gel, a hydrogel, and a conductive adhesive layer.

Illustrative Embodiment 80. The transducer array of illustrative embodiment 79, wherein the interface member has a first height less than a second height of the at least one protrusion.

Illustrative Embodiment 81. The transducer array of illustrative embodiment 65, wherein the support substrate comprises a second substrate surface opposite to the first substrate surface, and wherein the transducer array further comprises an interface member disposed between the first electrode surface of the electrode and the second substrate surface of the support substrate, wherein the interface member is one or more of a gel, a hydrogel, and a conductive adhesive layer.

Illustrative Embodiment 82. The transducer array of illustrative embodiment 65, further comprising a conductive material and wherein the at least one protrusion is either a needle or microneedle having a hollow core, the hollow core having the conductive material disposed therein.

Illustrative Embodiment 83. A method, comprising:
placing a transfer member at a target location on a patient such that at least one microneedle of the transfer member pierces skin of the patient;
interfacing an electrode with the patient; and
passing a tumor treating field through the electrode and the at least one microneedle of the transfer member, the tumor treating field having a frequency in a range between 50 kHz-1 MHz.

Illustrative Embodiment 84. An electrode element, comprising:
a transfer member having a support substrate constructed of a conductive material and a plurality of protrusions extending from a surface of the support substrate, the plurality of protrusions being microneedles,
an electrode being the support substrate of the transfer member whereby the transfer member and the electrode are a unitary structure; and
an interface element positioned between the microneedles.

From the above description, it is clear that the inventive concepts disclosed and claimed herein are well adapted to carry out the objects and to attain the advantages mentioned herein, as well as those inherent in the disclosure. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features and steps are recited in the claims, Illustrative embodiments, and/or disclosed in the specification, these combinations are not intended to limit the disclosure. In fact, many of these features and steps may be combined in ways not specifically recited in the claims, Illustrative embodiments, and/or disclosed in the specification. Although each dependent claim listed below may directly depend on only one other claim, the disclosure includes each dependent claim in combination with every other claim in the claim set.

Similarly, although each illustrative embodiment listed above may directly depend on only one other illustrative embodiment, the disclosure includes each illustrative embodiment in combination with every other illustrative embodiment in the set of illustrative embodiments for each mode of the inventive concepts disclosed herein.

No element, act, or instruction used in the present application should be construed as critical or essential to the disclosure unless explicitly described as such outside of the preferred embodiment. Further, the phrase “based on” is intended to mean “based, at least in part, on” unless explicitly stated otherwise.

What is claimed is:

1. A transducer array, comprising:
an electrode having a first skin-facing side and an opposing outwardly-facing second side, the electrode comprising a first electrode surface on the first skin-facing side, and a second electrode surface on the opposing outwardly-facing second side; and
a transfer member comprising a support substrate having a first substrate surface on a first skin-facing side, wherein the support substrate is located on the first skin-facing side of the electrode and wherein the transfer member further comprises at least one protrusion extending in a skin-facing direction from the first substrate surface and is configured to pierce a patient at a target area, the at least one protrusion having a base portion and an end portion.

2. The transducer array of claim 1, wherein the transfer member is constructed of a flexible material configured to contour to a shape of the target area.

3. The transducer array of claim 1, wherein the transfer member is constructed of a conductive material.

4. The transducer array of claim 1, wherein the at least one protrusion includes one or more of a needle and a microneedle, wherein the needle or microneedle has an elongated cone-shape or is a slender rodlike instrument with the end portion forming a point and having a height selected to pierce through an epidermis layer of a patient's skin, but to not pierce so far as to reach the patient's nerves.

5. The transducer array of claim 1, wherein the support substrate is the electrode whereby the electrode and the transfer member are a unitary structure.

6. The transducer array of claim 1, further comprising a dielectric material positioned between a first skin-facing side of the electrode and the first substrate surface of the transfer member.

7. The transducer array of claim 1, wherein the support substrate comprises a second substrate surface opposite to the first substrate surface, and wherein the transfer member further comprises a plurality of voids extending from the second substrate surface to the first substrate surface.

8. The transducer array of claim 7, wherein one or more of the plurality of voids is sized such that one or more of a gel, a hydrogel, and a conductive adhesive can pass through the one or more of the plurality of voids.

9. The transducer array of claim 1, wherein the end portion of the at least one protrusion is constructed of, or is embedded with, a salt.

10. The transducer array of claim 1, wherein the at least one protrusion further comprises a plurality of protrusions, and wherein the support substrate further comprises a second substrate surface, and wherein the transfer member further comprises a plurality of voids extending from the second substrate surface to the first substrate surface, the plurality of voids disposed between pairs of the plurality of protrusions.

11. The transducer array of claim 10, wherein the support substrate further comprises an inner portion and an outer portion, and wherein the plurality of protrusions is disposed within either the inner portion or the outer portion of the support substrate.

12. The transducer array of claim 10, wherein the support substrate further comprises an inner portion and an outer portion, wherein the plurality of protrusions is disposed within both the inner portion and the outer portion of the support substrate, and wherein a first density of the plurality of protrusions disposed within the inner portion of the transfer member differs from a second density of the plurality of protrusions disposed within the outer portion of the support substrate.

13. The transducer array of claim 1, wherein the at least one protrusion extending from the support substrate includes a first protrusion and a second protrusion, and wherein the first protrusion extends at a first angle relative to the electrode and the second protrusion extends at a second angle relative to the electrode, and wherein the first angle is different from the second angle.

14. The transducer array of claim 1, further comprising an interface member disposed against the first substrate surface of the support substrate.

15. The transducer array of claim 14, wherein the interface member is one or more of a gel, a hydrogel, and a conductive adhesive layer.

16. The transducer array of claim 15, wherein the interface member has a first height less than a second height of the at least one protrusion.

17. The transducer array of claim 1, wherein the support substrate comprises a second substrate surface opposite to the first substrate surface, and wherein the transducer array further comprises an interface member disposed between the first electrode surface of the electrode and the second substrate surface of the support substrate, wherein the interface member is one or more of a gel, a hydrogel, and a conductive adhesive layer.

18. The transducer array of claim 1, further comprising a conductive material and wherein the at least one protrusion is either a needle or microneedle having a hollow core, the hollow core having the conductive material disposed therein.

19. A method, comprising:
placing a transfer member at a target location on a patient such that at least one microneedle of the transfer member pierces skin of the patient;
interfacing an electrode with the patient; and
passing a tumor treating field through the electrode and the at least one microneedle of the transfer member, the tumor treating field having a frequency in a range between 50 kHz-1 MHz.

20. An electrode element, comprising:
a transfer member having a support substrate constructed of a conductive material and a plurality of protrusions extending from a surface of the support substrate, the plurality of protrusions being microneedles,
an electrode being the support substrate of the transfer member whereby the transfer member and the electrode are a unitary structure; and
an interface element positioned between the microneedles.
